(19) United States
(12) Patent Application Publication
(10) Pub. No.: US 2026/0007353 A1
(43) Pub. Date: Jan. 8, 2026

(54) METHOD AND SYSTEM TO IDENTIFY ATRIAL FIBRILLATION BASED ON DOMINANT ROTATIONAL DIRECTION OF INTERACTING ROTATIONAL ACTIVITIES

(71) Applicant: Northwestern University, Evanston, IL (US)

(72) Inventor: Markus Rottmann, Chicago, IL (US)

(21) Appl. No.: 19/259,336

(22) Filed: Jul. 3, 2025

Related U.S. Application Data
(60) Provisional application No. 63/667,221, filed on Jul. 3, 2024.

(51) Int. Cl. A61B 5/361; A61B 5/366; A61B 18/00; A61B 18/14

(52) U.S. Cl. CPC A61B 5/361; A61B 5/366; A61B 18/1492; A61B 2018/00351; A61B 2018/00577; A61B 2018/00642; A61B 2018/00839

(57) ABSTRACT

A system to identify atrial fibrillation includes an electrode array that senses data from an atrium of a heart. The system also includes a computing device operatively coupled to the electrode array. The computing device includes a processor configured to identify, based on the sensed data from the electrode array, one or more first locations of the atrium at which a dominant direction of reentry is clockwise. The processor is also configured to identify, based on the sensed data from the electrode array, one or more second locations of the atrium at which a dominant direction of reentry is anti-clockwise. The processor is also configured to identify, based on analysis of the one or more first locations and the one or more second locations, an intersection point, where the intersection point is a location of the atrium at which there is both a dominant clockwise reentry and a dominant anti-clockwise reentry. The processor is further configured to determine a target location for treatment based on the intersection point.

### CROSS-REFERENCE TO RELATED APPLICATION

The present application claims the priority benefit of U.S. Provisional Patent App. No. 63/667,221 filed on Jul. 3, 2024, the entire disclosure of which is incorporated by reference herein.

### BACKGROUND

Atrial fibrillation (AF) refers to a quivering or irregular heartbeat that can cause immediate symptoms such as heart palpitations, chest pain, fatigue, shortness of breath, dizziness, and overall weakness. Atrial fibrillation can also result in various long term health issues such as blood clots, heart failure, stroke, etc. During atrial fibrillation, the atrial (upper) chambers of the heart beat irregularly, which prevents normal blood flow through the (lower) ventricles.

### SUMMARY

An illustrative system to identify atrial fibrillation includes an electrode array that senses data from an atrium of a heart. The system also includes a computing device operatively coupled to the electrode array. The computing device includes a processor configured to identify, based on the sensed data from the electrode array, one or more first locations of the atrium at which a dominant direction of reentry is clockwise. The processor is also configured to identify, based on the sensed data from the electrode array, one or more second locations of the atrium at which a dominant direction of reentry is anti-clockwise. The processor is also configured to identify, based on analysis of the one or more first locations and the one or more second locations, an intersection point, where the intersection point is a location of the atrium at which there is both a dominant clockwise reentry and a dominant anti-clockwise reentry. The processor is further configured to determine a target location for treatment based on the intersection point.

In one embodiment, the processor further implements a treatment at the target location, and the treatment comprises an ablation. In another embodiment, an interstitial spacing of the electrode array is 2.5 millimeters or less. In another embodiment, the one or more first locations are identified based on a duration of time during which the dominant direction of rotation is clockwise. In another embodiment, the intersection point is identified based on a duration of time during which there is both the dominant clockwise reentry and the dominant anti-clockwise reentry.

In one embodiment, the processor generates one or more heat maps based on the sensed data, and wherein the one or more first locations and the one or more second locations are identified based on the one or more heat maps. In another embodiment, the processor is configured to determine reentry percentage over time based at least in part on a correlation between cycle length measurements and reentry percentage. In another embodiment, the processor is configured to determine a size including a length and a width of one or more clockwise reentries and one or more anti-clockwise reentries. In one embodiment, the processor is configured to determine a percentage of reentry degree completeness, where 100% is associated with 360 degrees.

In one embodiment, the processor is configured to determine and visualize a length of a block that blocks atrial activation. In another embodiment, the processor is configured to determine and visualize a gap length of block lines that block atrial activation, where the block lines that block atrial fibrillation have greater than a 25 millisecond activation delay between electrodes with 2.5 millimeter electrode spacing. In another embodiment, the processor is configured to determine and visualize atrial regions with high factors of clockwise versus anti-clockwise reentries and atrial regions with high factors of anti-clockwise versus clockwise reentries over time, wherein the high factors are greater than five factors. In another embodiment, the processor is configured to determine reentry percentage over time based at least in part on a correlation between activation delay between neighboring electrodes and reentry percentage. In another embodiment, the processor is configured to determine reentry percentage over time based at least in part on a correlation between peak-to-peak amplitude and reentry percentage.

An illustrative method of identifying atrial fibrillation includes receiving, at a memory of a computing device, data from an electrode array, where the data originates from an atrium of a heart. The method also includes identifying, by a processor of the computing device and based on the sensed data from the electrode array, one or more first locations of the atrium at which a dominant direction of reentry is clockwise. The method also includes identifying, by the processor and based on the sensed data from the electrode array, one or more second locations of the atrium at which a dominant direction of reentry is anti-clockwise. The method also includes identifying, by the processor and based on analysis of the one or more first locations and the one or more second locations, an intersection point, where the intersection point is a location of the atrium at which there is both a dominant clockwise reentry and a dominant anti-clockwise reentry. The method further includes determining, by the processor, a target location for treatment of the atrial fibrillation based on the intersection point.

In one embodiment, the method includes controlling, by the processor, a device to perform an ablation at the target location to treat the atrial fibrillation. In another embodiment, the method includes identifying the one or more first locations based on a duration of time during which the dominant direction of rotation is clockwise, where the duration of time is 10 seconds or less. In another embodiment, the method includes identifying the intersection point based at least in part on identification of a simultaneous occurrence of the dominant clockwise reentry and the dominant anti-clockwise reentry. In another embodiment, the method includes generating, by the processor, one or more heat maps based on the sensed data, and wherein the one or more first locations and the one or more second locations are identified based on the one or more heat maps. In another embodiment, the method includes determining, by the processor, reentry percentage over time based at least in part on activation delay between neighboring electrodes in the electrode array.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts a high density electrode plaque for use in monitoring a heart that has AF in accordance with an illustrative embodiment.

FIG. 1B depicts the various regions of the heart (i.e., PLA, LAFW, LAA, PRA, RAFW, and RAA) that are analyzed using the electrode configuration of FIG. 1A in accordance with an illustrative embodiment.

FIG. 2 is a flow diagram illustrating operations performed to determine predominant rotation direction of rotational activities in the atria in accordance with an illustrative embodiment.

FIG. 3A depicts various rotational activities detected in each of the 6 atrial regions in accordance with an illustrative embodiment.

FIG. 3B depicts the observed reentry percentage values in accordance with an illustrative embodiment.

FIG. 3C depicts cycle length for each of the six atrial regions in accordance with an illustrative embodiment.

FIG. 3D depicts reentry percentage versus cycle length in accordance with an illustrative embodiment.

FIG. 3E depicts observed PLA clockwise reentry in accordance with an illustrative embodiment.

FIG. 3F depicts observed anti-clockwise reentry in accordance with an illustrative embodiment.

FIG. 3G depicts observed clockwise reentry percentage over a 10 second time period in accordance with an illustrative embodiment.

FIG. 3H depicts observed anti-clockwise reentry percentage over a 10 second time period in accordance with an illustrative embodiment.

FIG. 3I depicts observed clockwise and anti-clockwise reentry percentage over a 10 second time period in accordance with an illustrative embodiment.

FIG. 3J depicts clockwise and anti-clockwise reentry percentages for each atrial region in accordance with an illustrative embodiment.

FIG. 4 depicts a summary of study results for clockwise versus anti-clockwise reentry percentage over a 10 second period in accordance with an illustrative embodiment.

FIG. 5A depicts clockwise reentry rotation direction in accordance with an illustrative embodiment.

FIG. 5B depict anti-clockwise reentry rotation in accordance with an illustrative embodiment.

FIG. 5C depicts the intersection point between clockwise and anti-clockwise rotation reentries in accordance with an illustrative embodiment.

FIG. 5D depicts examples of 100% reentry completeness and 75% reentry completeness in accordance with an illustrative embodiment.

FIG. 5E depicts 75% and 100% reentry completeness around block lines in accordance with an illustrative embodiment.

FIG. 6A is a first example of test results depicting clockwise and anti-clockwise reentry percentage over 10 seconds, the sum of time gradient between electrodes (2.5 mm spacing), and average voltage in accordance with an illustrative embodiment.

FIG. 6B is a second example of test results depicting clockwise and anti-clockwise reentry percentage over 10 seconds, the sum of time gradient between electrodes (2.5 mm spacing), and average voltage in accordance with an illustrative embodiment.

FIG. 6C depicts a third example of test results depicting clockwise and anti-clockwise reentry percentage over 10 seconds, the sum of time gradient between electrodes (2.5 mm spacing), and average voltage in accordance with an illustrative embodiment.

FIG. 6D depicts a fourth example of test results depicting clockwise and anti-clockwise reentry percentage over 10 seconds, the sum of time gradient between electrodes (2.5 mm spacing), and average voltage in accordance with an illustrative embodiment.

FIG. 6E depicts a fifth example of test results depicting clockwise and anti-clockwise reentry percentage over 10 seconds, the sum of time gradient between electrodes (2.5 mm spacing), and average voltage in accordance with an illustrative embodiment.

FIG. 6F depicts a sixth example of test results depicting clockwise and anti-clockwise reentry percentage over 10 seconds, the sum of time gradient between electrodes (2.5 mm spacing), and average voltage in accordance with an illustrative embodiment.

FIG. 6G depicts a seventh example of test results depicting clockwise and anti-clockwise reentry percentage over 10 seconds, the sum of time gradient between electrodes (2.5 mm spacing), and average voltage in accordance with an illustrative embodiment.

FIG. 6H depicts an eighth example of test results depicting clockwise and anti-clockwise reentry percentage over 10 seconds, the sum of time gradient between electrodes (2.5 mm spacing), and average voltage in accordance with an illustrative embodiment.

FIG. 6I depicts anti-clockwise rotational activity over time in accordance with an illustrative embodiment.

FIG. 6J depicts factor clockwise versus anti-clockwise rotational activity over time in a range up to 100% in accordance with an illustrative embodiment.

FIG. 6K depicts a strong correlation between activation delay among 2 neighboring (i.e., adjacent) electrodes (2.5 mm distance) and observed percentage over time in accordance with an illustrative embodiment.

FIG. 7 depicts heat maps of slow conduction zones (activation delay over time (10 seconds)), and average voltage over time (10 seconds) in sinus rhythm/paced rhythm (upper panel) and in AF (lower panel) and showing similar located slow conduction zones (marked with ovals) in accordance with an illustrative embodiment.

FIG. 8A depicts a 3D local activation time map in accordance with an illustrative embodiment.

FIG. 8B depicts that ablation at a slowest reentry site terminated persistent AF in accordance with an illustrative embodiment.

FIG. 8C depicts low voltage at the slowest reentry site in accordance with an illustrative embodiment.

FIG. 8D depicts the lowest reentry site at the border between high and low entropy in accordance with an illustrative embodiment.

FIG. 8E depicts how reentry and focal drivers had significantly lower voltage (critical AF zones) compared to not critical AF zones without reentry or focal drivers in accordance with an illustrative embodiment.

FIG. 8F depicts reentry frequency at low voltage and slow conduction in dogs, correlation reentry with slow conduction and voltage, and correlation of slow conduction and voltage in accordance with an illustrative embodiment.

FIG. 9A is a chart that depicts the number of ablated mapping plaque quadrants with slowest reentry in the atrial regions and number of performed control quadrant ablations that lead to termination in 2 cases in accordance with an illustrative embodiment.

FIG. 9B depicts that ablation at slow CV regions reduced observed reentry over time percentage in all regions in accordance with an illustrative embodiment.

FIG. 9C depicts pulmonary vein isolation (PVI) reduced observed reentry over time percentage in all regions but not in the LAA and RAFW in accordance with an illustrative embodiment.

FIG. 10 depicts how block lines shift cycle by cycle but are similarly located and parallel over time in accordance with an illustrative embodiment.

FIG. 11A depicts Masson's trichrome stained tissue sections in the six atrial regions in accordance with an illustrative embodiment.

FIG. 11B depicts the degree of focal fibrosis or fat in all atrial regions in both atria in accordance with an illustrative embodiment.

FIG. 11C depicts the highest number of myocyte bundle nodal points that were detected in the RAA, LAA, and PLA in accordance with an illustrative embodiment.

FIG. 11D depicts correlation of the degree of fibrosis/fat with detected slow conduction zones (activation delay zones>10 ms) (R=0.7, P<0.05) (N=35) in accordance with an illustrative embodiment.

FIG. 11E depicts correlation of myocyte bundle nodal points with the stability of rotational activity (R=0.6, P<0.05) (N=35) in accordance with an illustrative embodiment.

FIG. 11F depicts positive correlation between the observed rotational activity over time (%) and the degree of fibrosis in all atrial regions in accordance with an illustrative embodiment.

FIG. 11G depicts positive correlation between the number of fiber bundle nodal points and the observed rotational activities over time in all regions but not the left and right atrial appendage in accordance with an illustrative embodiment.

FIG. 12A depicts normal PLA versus AF PLA in accordance with an illustrative embodiment.

FIG. 12B depicts dense fibrosis versus interstitial fibrosis in accordance with an illustrative embodiment.

FIG. 12C depicts voltage, planar wave slow CV, and reentry slow CV in accordance with an illustrative embodiment.

FIG. 13 includes tables comparing a control case versus an AF case in accordance with an illustrative embodiment.

FIG. 14 depicts a computing device in direct or indirect communication with an electrode array in accordance with an illustrative embodiment.

### DETAILED DESCRIPTION

Atrial fibrillation (AF) is the most common form of heart disease. In the United States, it is estimated that 1 in 25 people at the age of 60 and 1 in 10 people at the age of 80 have atrial fibrillation, with over 6 million individuals currently suffering from the condition. By 2033, it is estimated that there will be more than 10 million in the aging population with AF. It is also estimated that AF occurs in 0.4% of the general population, 4% of the hospital population, and 40% of patients with heart failure. In addition to its effects on the heart, atrial fibrillation is also a major cause of stroke, with an estimated 15% of all strokes occurring as a result of AF. As a result, there is a huge economic impact related to AF, costing billions of dollars annually in both direct and indirect costs.

Given the number of individuals affected and the overall economic impact, the successful treatment of atrial fibrillation is an important challenge in cardiovascular medicine. Unfortunately, traditional antiarrhythmic drugs and catheter ablation therapies (e.g., PVI, OI, FI, ShEn, CFAE, DF, etc.) have limited efficacy in the treatment of atrial fibrillation, likely because they are not targeting the underlying molecular and structural mechanisms of AF. For example, current drug treatments have less than 50% efficacy and can cause life-threatening arrhythmias. Ablation has only a roughly 70% of success in earlier stages age of AF and often requires repeat procedures. Ablation success is less than 50% in advanced persistent AF.

Thus, new systems and software algorithms are needed to optimize the clinical procedures used to treat AF. Rotational activities in the heart are established drivers of atrial fibrillation, but their mechanisms over time are complex and still not well understood. Although it is known that multiple rotational activities interact dynamically over time in both atria, the quantification of reentry rotation direction over time is still lacking. Described herein are novel systems and methods/algorithms for the detection and treatment of AF. More specifically, described herein are methods and systems to quantify the rotation direction and cycle length of clockwise and anti-clockwise rotational activities in the heart over time to help diagnose and treat all forms of AF.

The methods and systems described herein are based on the results of an AF study conducted on canines. To conduct the study, AF was induced in 43 dogs by rapid atrial pacing (RAP) for 3 to 14 weeks. Before all the procedures, animals were premedicated with acepromazine (0.01-0.02 mg/kg) and induced with propofol (3-7 mg/kg). All experiments were performed under general anesthesia (inhaled) with isoflurane (1%-3%). Adequacy of anesthesia was assessed by toe pinch and palpebral reflex.

For pacemaker insertion, the right jugular vein was accessed by direct cutdown and ligated distally. A bipolar screw-in Medtronic pacing lead was inserted through an incision in the right jugular vein. The tip of the lead was fluoroscopically placed and fixed in the right atrial appendage after confirming an adequate capture threshold (<0.5 mV with a pulse width of 0.4 ms). The proximal end of the pacing lead was connected to a custom-modified Medtronic programmable pulse generator that was subsequently implanted in a subcutaneous pocket in the neck. After all the incisions were closed, the dogs were allowed to recover from anesthesia and were returned to the animal quarters.

After induction of persistent AF, high-density open chest mapping was performed on the epicardium of each test subject using the UnEmap mapping system (University of Auckland, Auckland, New Zealand). Six atrial regions were mapped in both atria in 31 dogs with AF. The UnEmap mapping system was used with a triangular mapping plaque and records 117 bipolar electrogram signals (1 kHz sampling rate, 130 electrodes, interelectrode distance of 2.5 millimeters (mm)). In alternative embodiments, a different sampling rate (e.g., 2 kHz, 5 kHz, etc.), number of electrodes (e.g., 120, 140, 150, 175, etc.), and/or distance between electrodes (e.g., 0.5 mm, 1 mm, 2 mm, etc.) may be used. Bipolar electrograms were recorded from 6 different regions in both atria: posterior left atrium (PLA), left atrial free wall (LAFW), left atrial appendage (LAA), posterior right atrium (PRA), right atrial free wall (RAFW, and right atrial appendage (RAA). The following AF signal characteristics were analyzed: dominant frequency (DF), cycle length (CL), and organization index (OI).

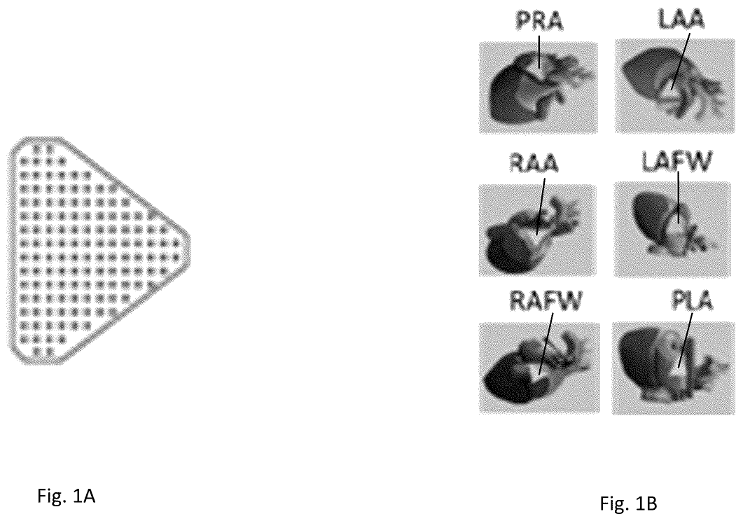

FIG. 1A depicts a high density electrode plaque for use in monitoring a heart that has AF in accordance with an illustrative embodiment. In this embodiment, there are 130 electrodes arranged in a triangular pattern, and the spacing in between electrodes is 2.5 mm. In alternative embodiments, a different number of electrodes may be used, such as 100, 120, 150, 200, etc. In another alternative embodiment, a different electrode spacing may be used, such as 1 mm, 2 mm, 3 mm, 4 mm, etc. FIG. 1B depicts the various regions of the heart (i.e., PLA, LAFW, LAA, PRA, RAFW, and RAA) that are analyzed using the electrode configuration of FIG. 1A in accordance with an illustrative embodiment. The electrode array can mounted/attached to the epicardium (i.e., outside of the heart) proximate to the atrial chambers in an illustrative embodiment. In alternative embodiments, an internal electrode catheter may be used to position the electrode array within the heart.

In an illustrative embodiment, the electrodes measure planar wave activation signals within the atrium. The planar waves can include and R-wave and an S-wave of a QRS complex. The QRS complex is a representation of the depolarization (i.e., electrical activation) of the heart's chambers. The QRS can be a combination of 3 waves, which include a Q wave (so-called if the first wave is a negative wave), an R wave (a positive wave, which may follow the Q wave), and an S wave (a negative wave that follows a positive wave). A QRS complex can be referred to as net positive if the sum of the waves which make up the QRS complex results in a positive value (wave) relative to a baseline. Similarly, a QRS complex can be referred to as net negative if the sum of the waves which make up the QRS complex results in a negative value (wave) relative to a baseline.

As part of the study, cycle length (CL) was calculated for each electrode signal. Additionally, rotational activities over time were automatically detected in local activation time maps using Matlab. In alternative embodiments, a different software or program may be used to detect the rotational activities and/or generate local activation time maps. The observed clockwise and anti-clockwise rotations were quantified over 10-second recordings in all atrial regions for each of the test subjects. For each electrode position, the factor of overserved clockwise vs anti-clockwise reentries (%) over time was calculated.

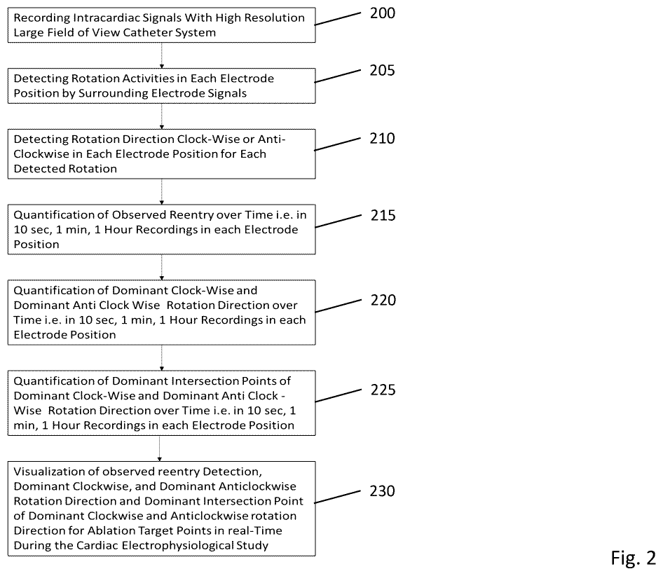

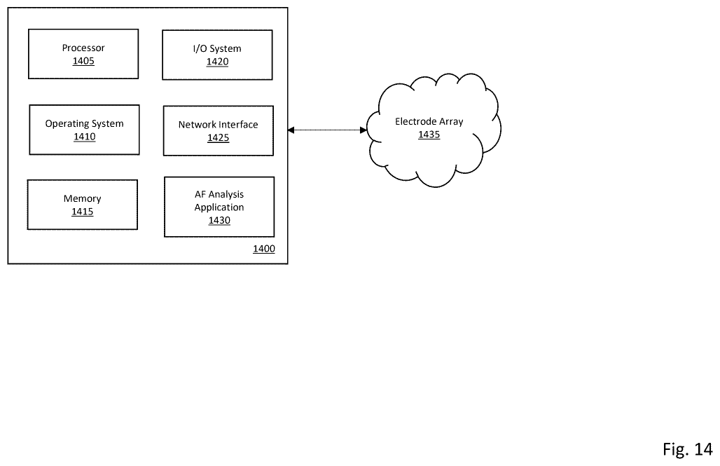

FIG. 2 is a flow diagram illustrating operations performed to determine predominant rotation direction of rotational activities in the atria in accordance with an illustrative embodiment. In alternative embodiments, fewer, additional, and/or different operations may be performed. Additionally, the use of a flow diagram is not meant to be limiting with respect to the order in which the operations are performed. In an operation 200, a large field of view catheter electrode system is used to obtain high resolution intracardiac signals. In an illustrative embodiment, the electrode array of FIG. 1A can be used to obtain the intracardiac signals. The signals can be received and recorded (i.e., stored) in a computing device for further processing, as discussed in more detail below with reference to FIG. 14. In an illustrative embodiment, the computing device can perform signal processing on the signals/data received from the electrode array, such as low pass filtering and high pass filtering to exclude extreme readings, noise filtering, verifying that signal amplitude falls within a desired range, detecting activation times, generating and quantifying activation time maps, etc.

In an operation 205, rotational activities are detected at each electrode position using surrounding electrode signals. Reentry rotational activities are important to study, as they are drivers for AF. As discussed herein, cycling time can also be considered. Cycling refers to the time in between 2 waveforms, and cycling time is negatively correlated. In practice, the fastest reentries are most often observed/detected, and these fast reentries are the most important drivers of AF, and are therefore the most likely to be associated with regions of interest at which ablation can help to prevent further AF.

In an operation 210, rotation direction (i.e., either clockwise or anti-clockwise) is determined at each electrode position for each detected rotation. In an operation 215, reentry over time is observed and quantified at each electrode position. In an illustrative embodiment, the time period can be 10 seconds(s), 1 minute, 1 hour, etc. In an operation 220, the dominant direction of rotation (i.e., either clockwise or anti-clockwise) is quantified over time at each of the electrode locations. The time period can be 10 s, 1 minute, 1 hour, etc. In an operation 225, any intersection points of the dominant clockwise and dominant anti-clockwise rotations are identified at each electrode position over the same time period. It has been determined that these intersection points contribute to AF, and are therefore important to identify for treatment. In an operation 230, the system identifies ablation target points based on the observed dominant directions of rotation, along with intersection point(s) of the dominant rotations in each direction.

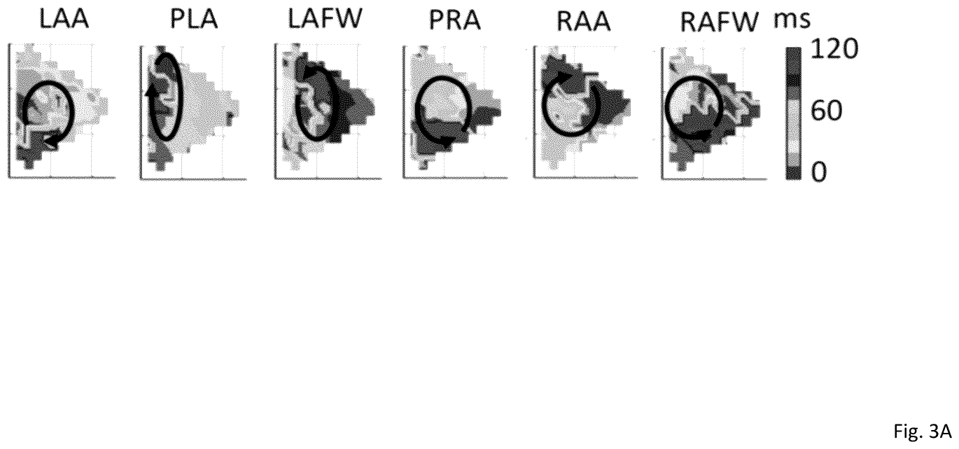

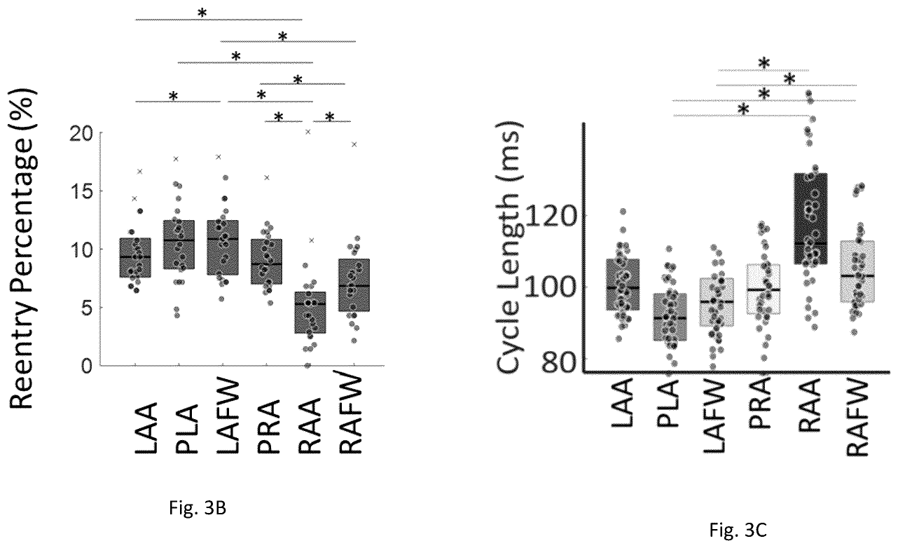

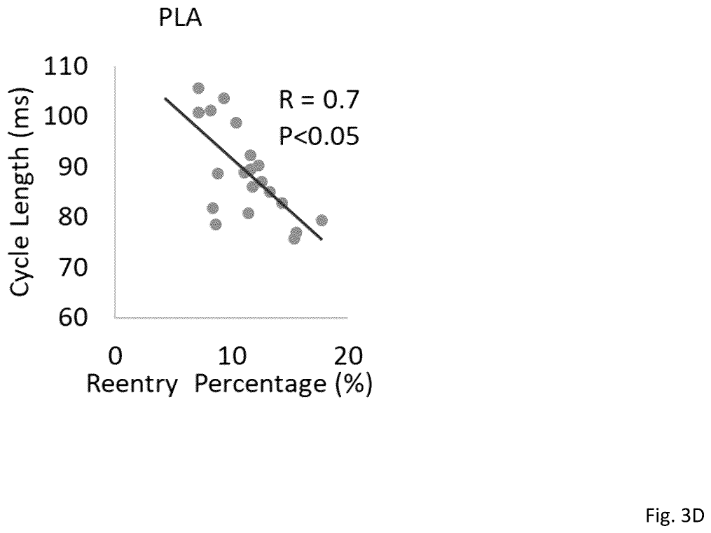

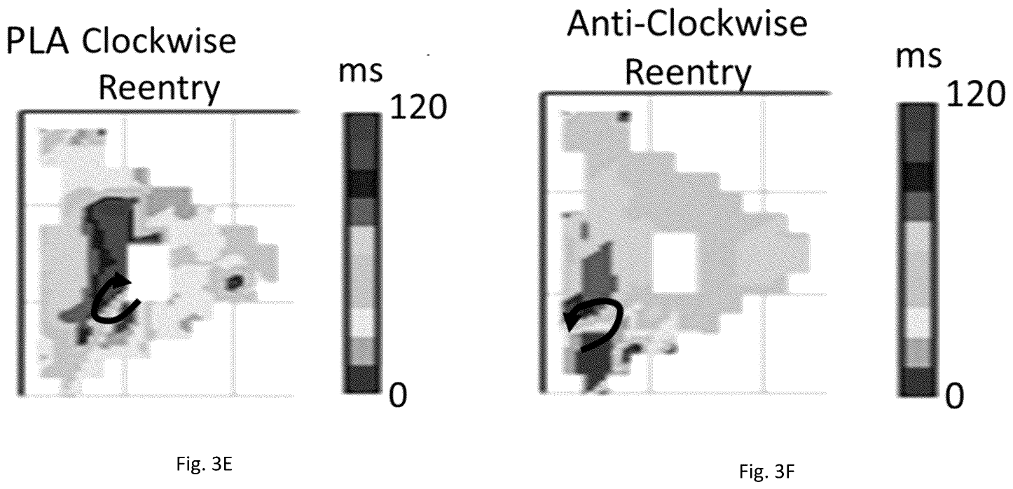

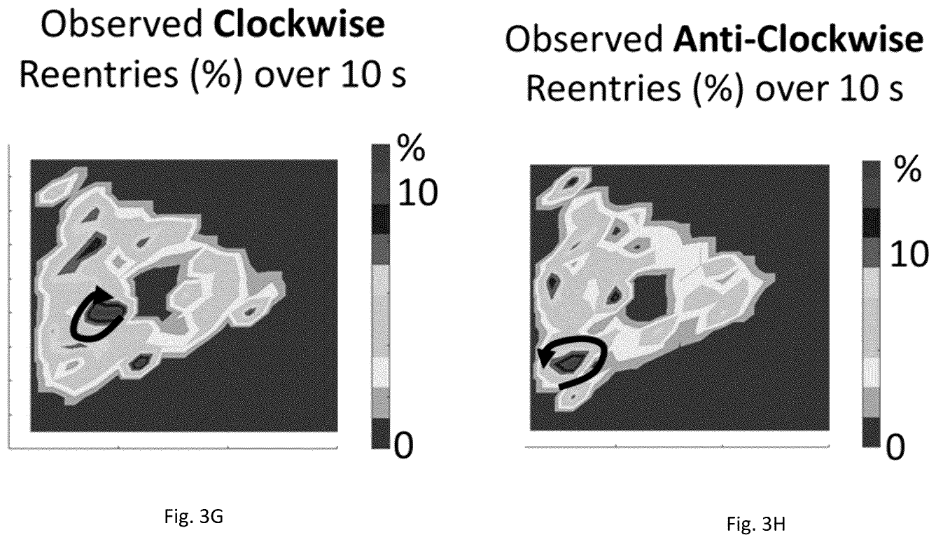

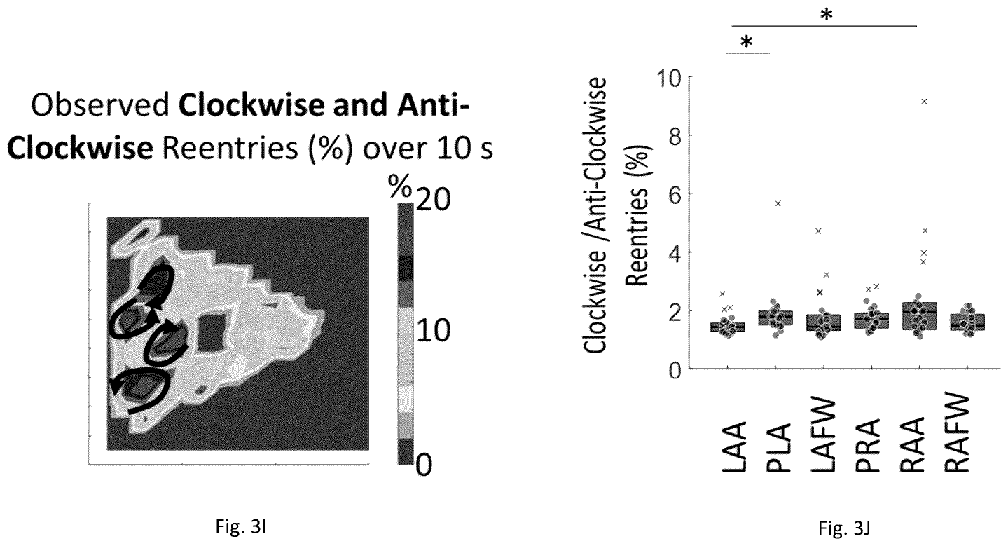

During the above-discussed study implementing the process of FIG. 2, multiple interacting rotational activities in persistent AF were detected in all six atrial regions. FIG. 3A depicts various rotational activities detected in each of the 6 atrial regions in accordance with an illustrative embodiment. The highest observed reentry percentage values over time were in the PLA and LAFW, and the lowest were in the right atrial appendages (12% vs 5%, P<0.05). FIG. 3B depicts the observed reentry percentage values in accordance with an illustrative embodiment. Cycle lengths were also analyzed. The shortest cycle length of rotational activity was in the PLA (CL 95 ms) compared to other atrial regions (P<0.05). FIG. 3C depicts cycle length for each of the six atrial regions in accordance with an illustrative embodiment. It was determined that the observed reentry percentage over time negatively correlated with CL (R=0.7, P<0.05) in atrial regions. FIG. 3D depicts reentry percentage versus cycle length in accordance with an illustrative embodiment.

Clockwise and anti-clockwise rotational activities were observed in the same atrial regions over time. FIG. 3E depicts observed PLA clockwise reentry in accordance with an illustrative embodiment. FIG. 3F depicts observed anti-clockwise reentry in accordance with an illustrative embodiment. It was observed that dominant clockwise and dominant anti-clockwise reentries closely interacted with each other over time. FIG. 3G depicts observed clockwise reentry percentage over a 10 second time period in accordance with an illustrative embodiment. FIG. 3H depicts observed anti-clockwise reentry percentage over a 10 second time period in accordance with an illustrative embodiment. FIG. 3I depicts observed clockwise and anti-clockwise reentry percentage over a 10 second time period in accordance with an illustrative embodiment. The factor of overserved clockwise vs anti-clockwise reentries over time (%) in all 6 atrial regions was 1.8±0.3 and was highest in the RAA with 2.3±1.7 (P<0.05) and in the PLA 1.9±0.9 (P<0.05). FIG. 3J depicts clockwise and anti-clockwise reentry percentages for each atrial region in accordance with an illustrative embodiment.

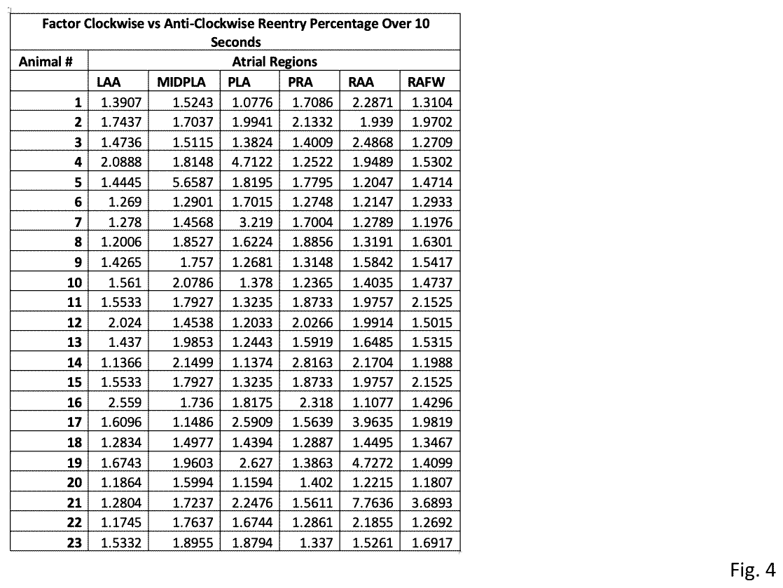

Thus, the study demonstrates that AF reentries in persistent AF have a nearly two-fold higher preferred rotation direction at the same tissue location over time in all atrial regions, and this preferred rotation direction is caused by underlying substrate characteristics. The real-time identification of the dominant reentry rotation direction during the cardiac electrophysiological study can be used as ablation target points at the isthmus between clockwise and anti-clockwise reentries outside the pulmonary veins. As a result, the proposed system can be used to identify ablation target points and to help eliminate the AF by performing treatment at the target points. FIG. 4 depicts a summary of study results for clockwise versus anti-clockwise reentry percentage over a 10 second period in accordance with an illustrative embodiment. Additional results from the study are discussed below.

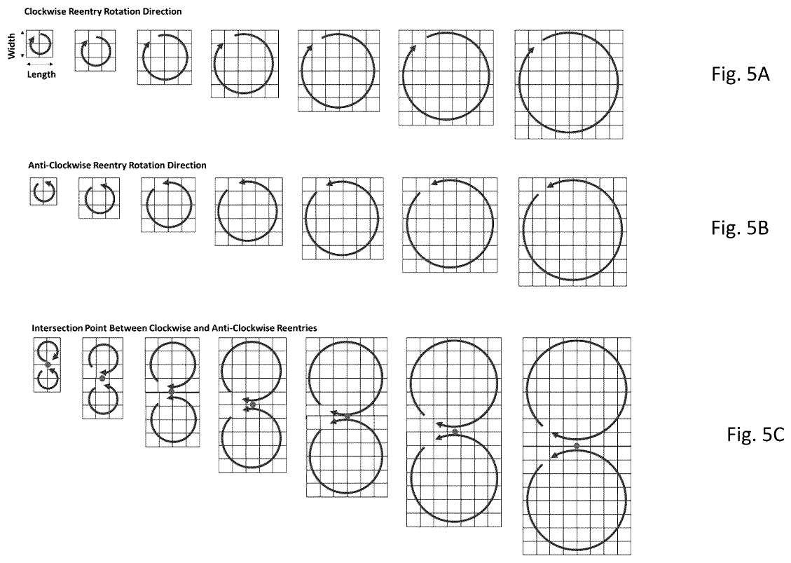

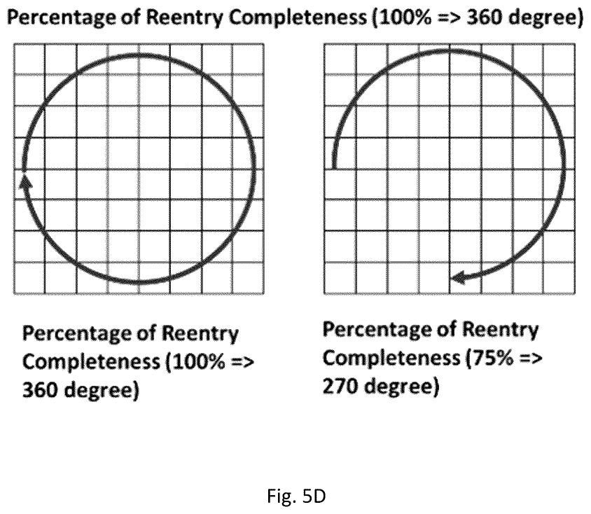

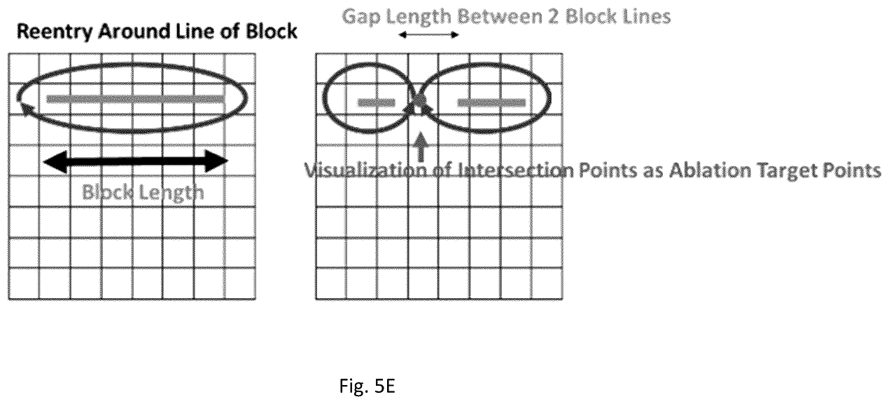

FIG. 5A depicts clockwise reentry rotation direction in accordance with an illustrative embodiment. FIG. 5B depict anti-clockwise reentry rotation in accordance with an illustrative embodiment. FIG. 5C depicts the intersection point between clockwise and anti-clockwise rotation reentries in accordance with an illustrative embodiment. FIG. 5D depicts examples of 100% reentry completeness and 75% reentry completeness in accordance with an illustrative embodiment. FIG. 5E depicts 75% and 100% reentry completeness around block lines in accordance with an illustrative embodiment. The reentry parameters considered with respect to FIG. 5 include rotational direction, reentry percentage over time, length of reentry, width of reentry, intersection points of clockwise and anti-clockwise reentries, observed intersection points of clockwise and anti-clockwise reentries over time, percentage of completeness of reentry (i.e. 360 degree rotation, 270 degree rotation, 300 degree rotation, 330 degree rotation), length and width of block lines (e.g., defined as >10 ms activation time delay within 2.5 mm), gap length and width between block lines, intersection points of clockwise and anti-clockwise reentries at block lines and gaps between block lines.

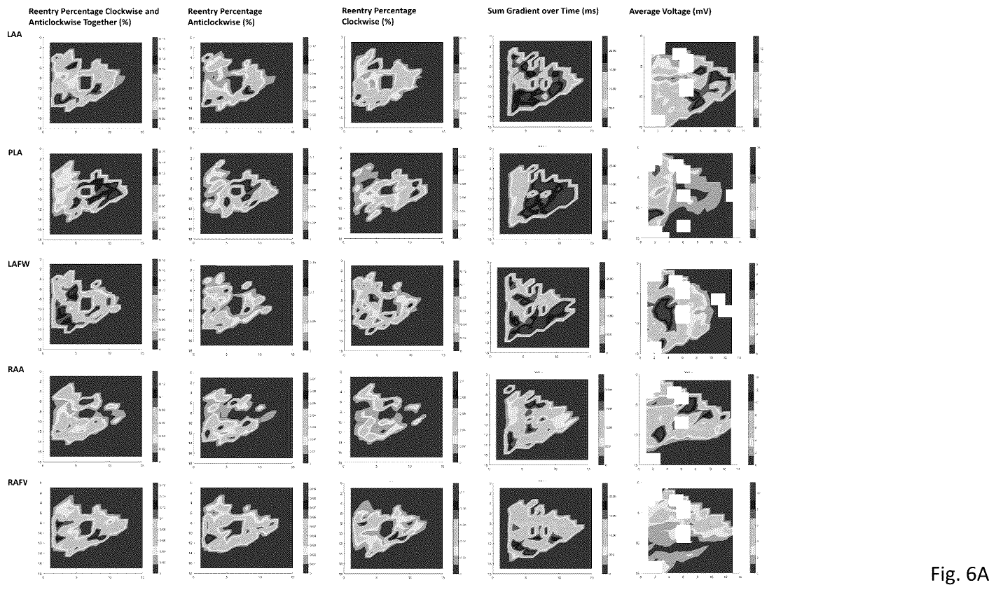

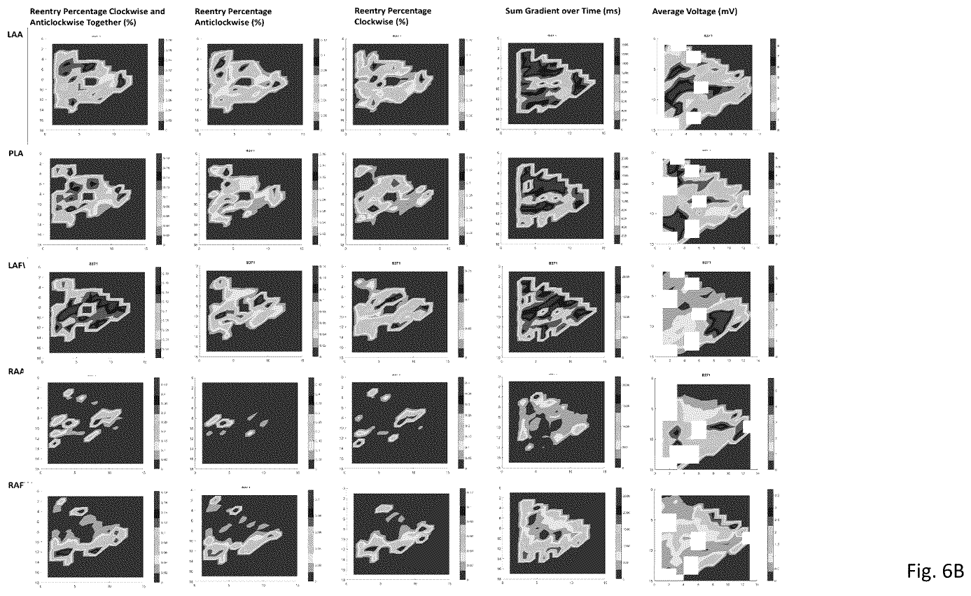

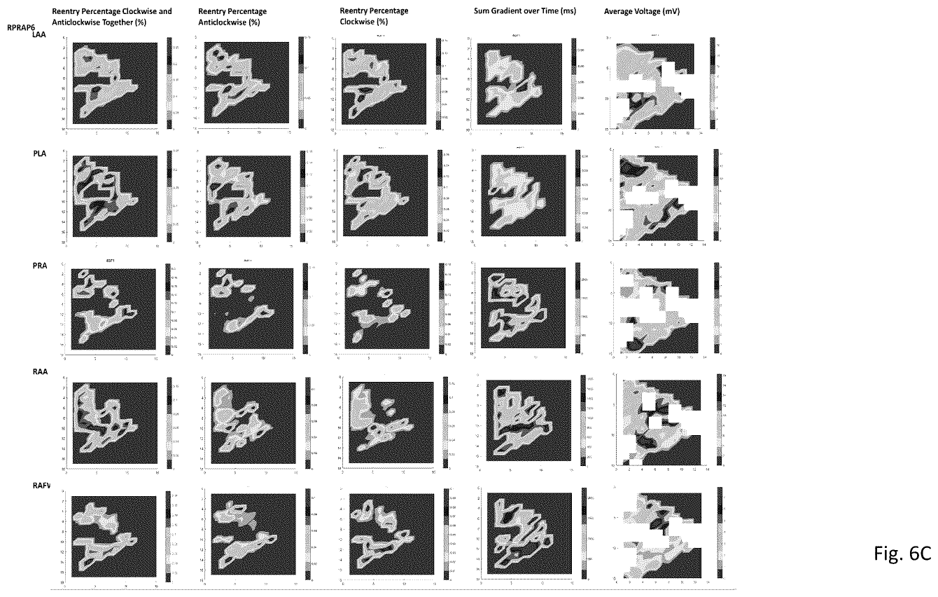

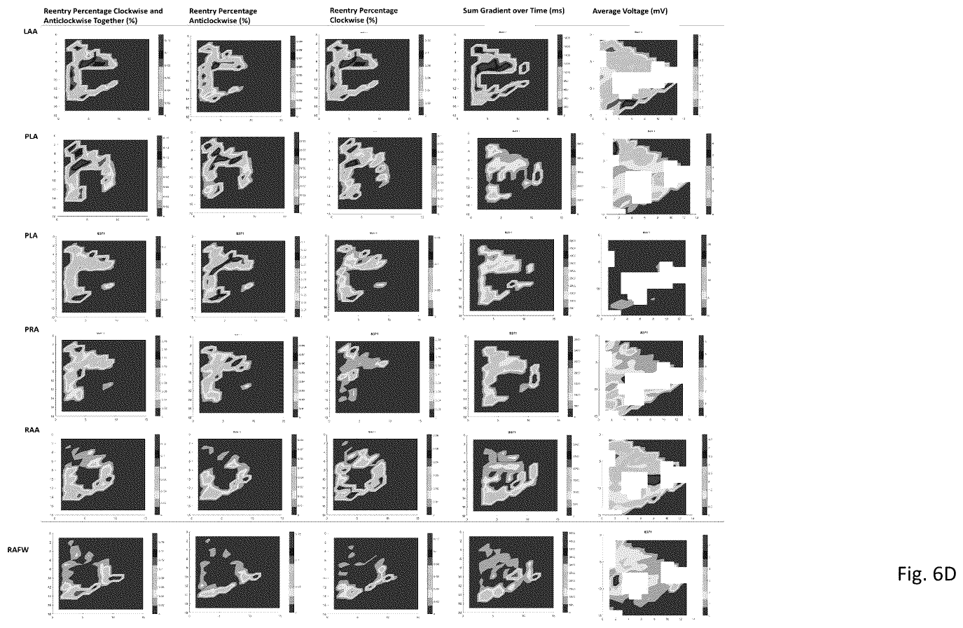

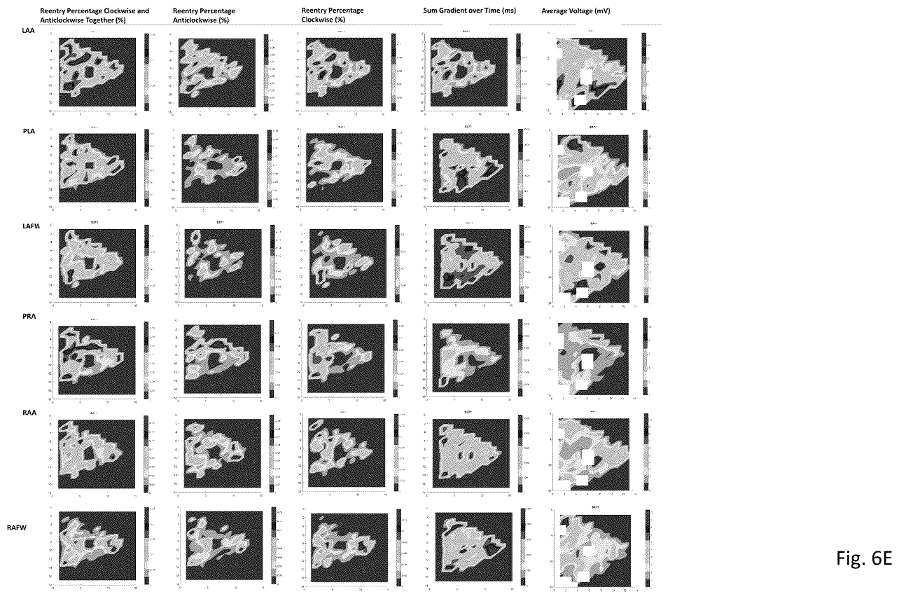

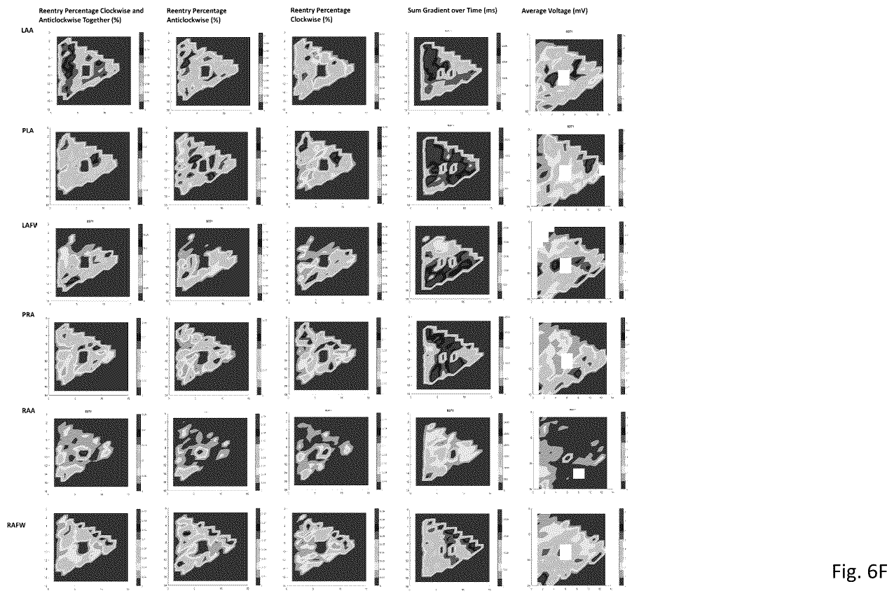

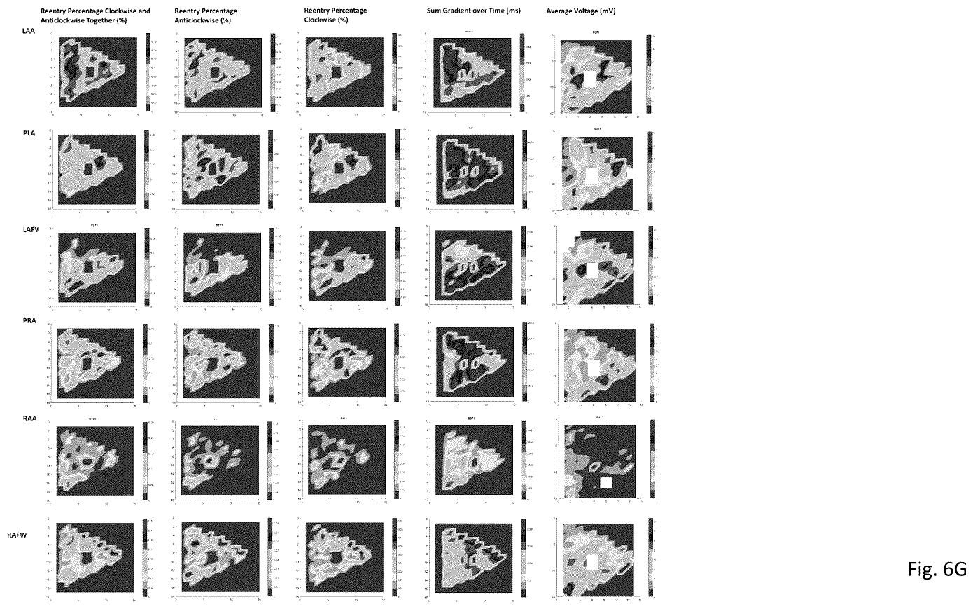

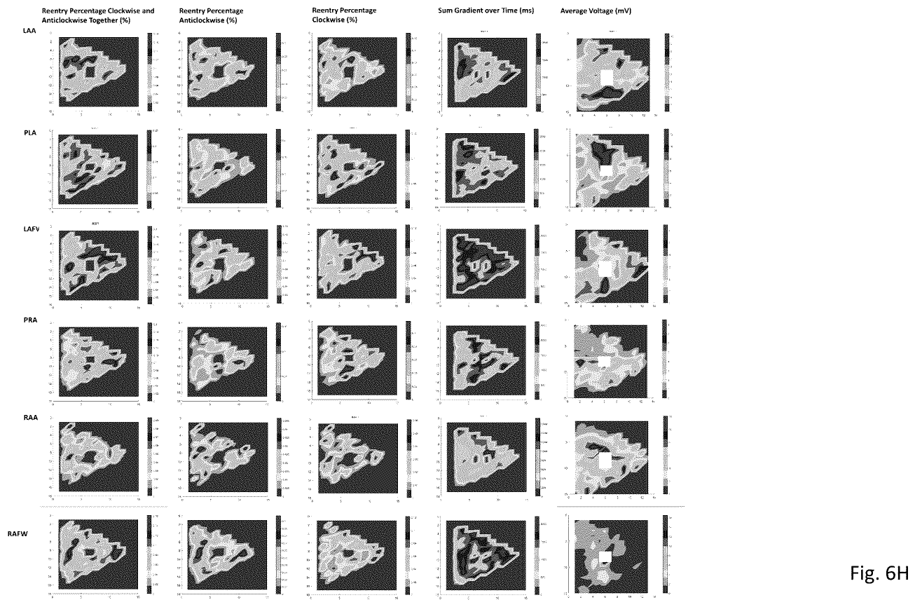

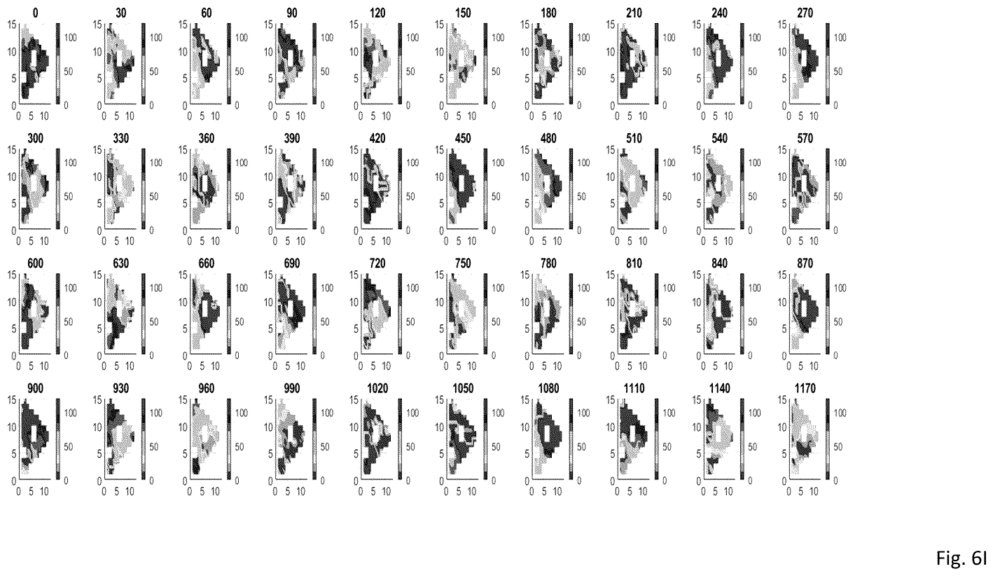

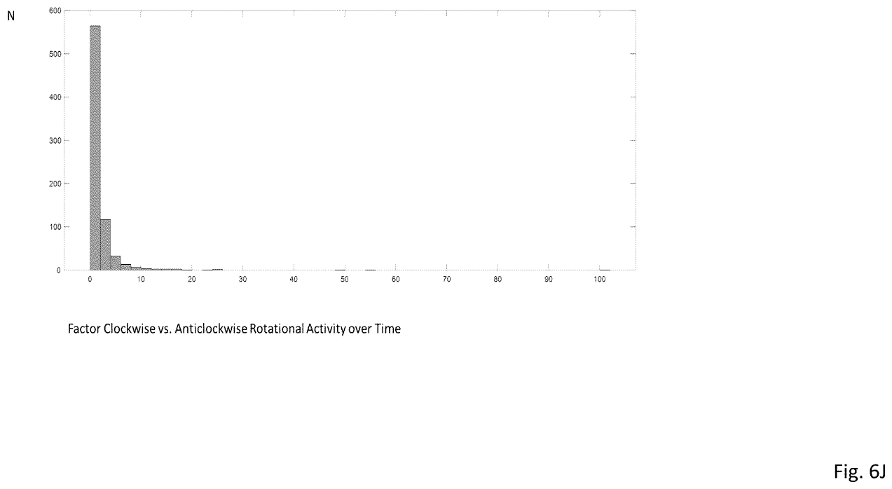

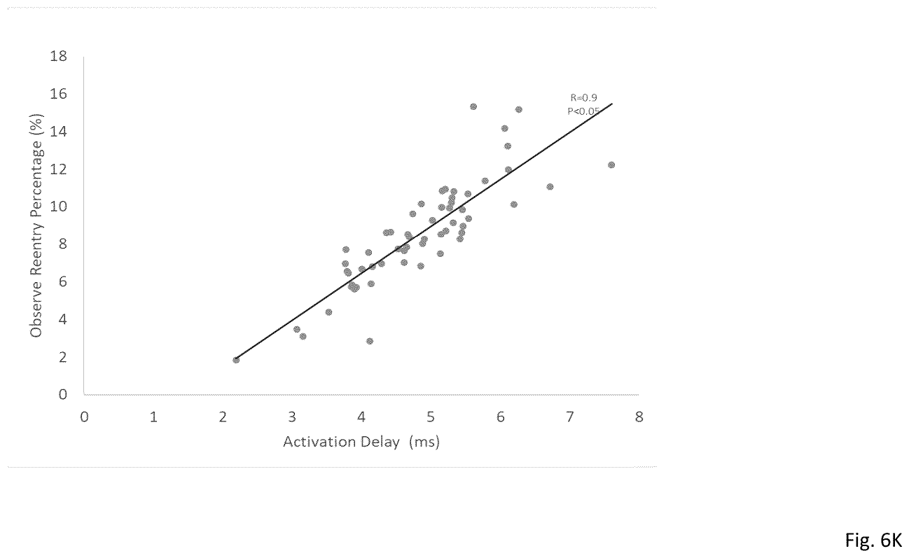

FIG. 6A is a first example of test results depicting clockwise and anti-clockwise reentry percentage over 10 seconds, the sum of time gradient between electrodes (2.5 mm spacing), and average voltage in accordance with an illustrative embodiment. FIG. 6B is a second example of test results depicting clockwise and anti-clockwise reentry percentage over 10 seconds, the sum of time gradient between electrodes (2.5 mm spacing), and average voltage in accordance with an illustrative embodiment. FIG. 6C depicts a third example of test results depicting clockwise and anti-clockwise reentry percentage over 10 seconds, the sum of time gradient between electrodes (2.5 mm spacing), and average voltage in accordance with an illustrative embodiment. FIG. 6D depicts a fourth example of test results depicting clockwise and anti-clockwise reentry percentage over 10 seconds, the sum of time gradient between electrodes (2.5 mm spacing), and average voltage in accordance with an illustrative embodiment. FIG. 6E depicts a fifth example of test results depicting clockwise and anti-clockwise reentry percentage over 10 seconds, the sum of time gradient between electrodes (2.5 mm spacing), and average voltage in accordance with an illustrative embodiment. FIG. 6F depicts a sixth example of test results depicting clockwise and anti-clockwise reentry percentage over 10 seconds, the sum of time gradient between electrodes (2.5 mm spacing), and average voltage in accordance with an illustrative embodiment. FIG. 6G depicts a seventh example of test results depicting clockwise and anti-clockwise reentry percentage over 10 seconds, the sum of time gradient between electrodes (2.5 mm spacing), and average voltage in accordance with an illustrative embodiment. FIG. 6H depicts an eighth example of test results depicting clockwise and anti-clockwise reentry percentage over 10 seconds, the sum of time gradient between electrodes (2.5 mm spacing), and average voltage in accordance with an illustrative embodiment. FIG. 6I depicts anti-clockwise rotational activity over time in accordance with an illustrative embodiment. FIG. 6J depicts factor clockwise versus anti-clockwise rotational activity over time in a range up to 100% in accordance with an illustrative embodiment. FIG. 6K depicts a strong correlation between activation delay among 2 neighboring (i.e., adjacent) electrodes (2.5 mm distance) and observed percentage over time in accordance with an illustrative embodiment.

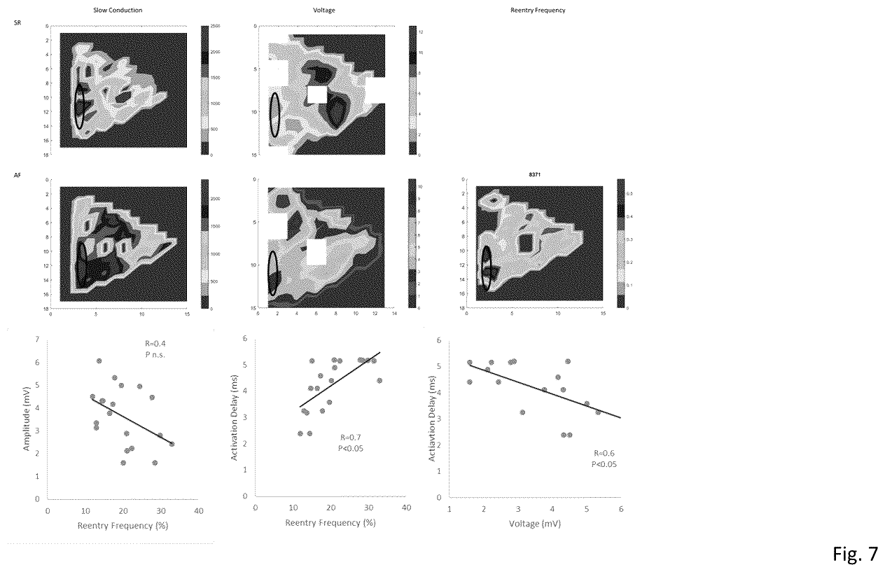

FIG. 7 depicts heatmaps of slow conduction zones (activation delay over time (10 seconds)), and average voltage over time (10 seconds) in sinus rhythm/paced rhythm (upper panel) and in AF (lower panel) and showing similar located slow conduction zones (marked with ovals) in accordance with an illustrative embodiment. Also often observed reentries over time were located in this similar location (marked in black). As also shown, correlation of observed reentries over time and amplitude was performed, correlation of observed reentries over time and slow conduction assessed as activation delay was performed, and correlation of voltage and slow conduction assessed as activation delay was performed.

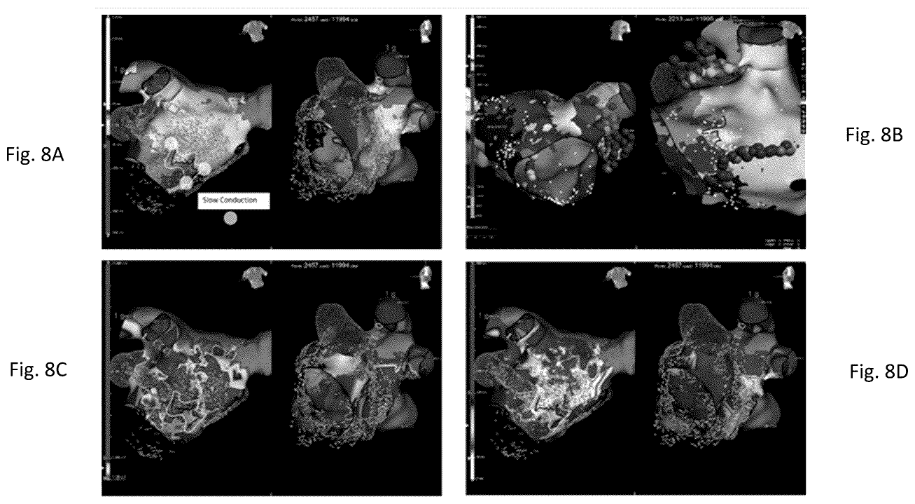

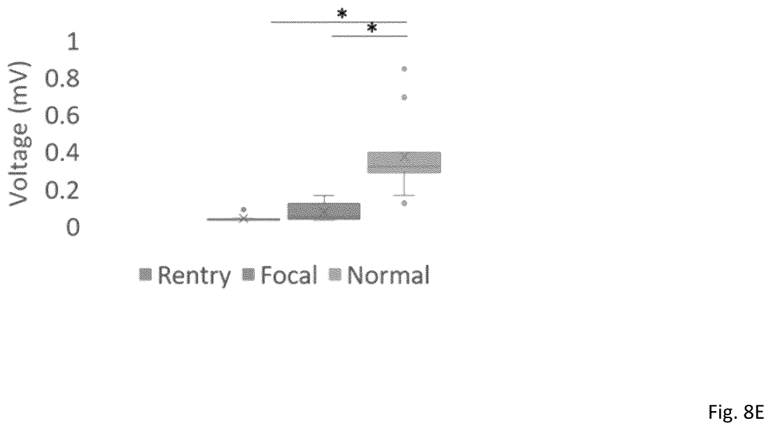

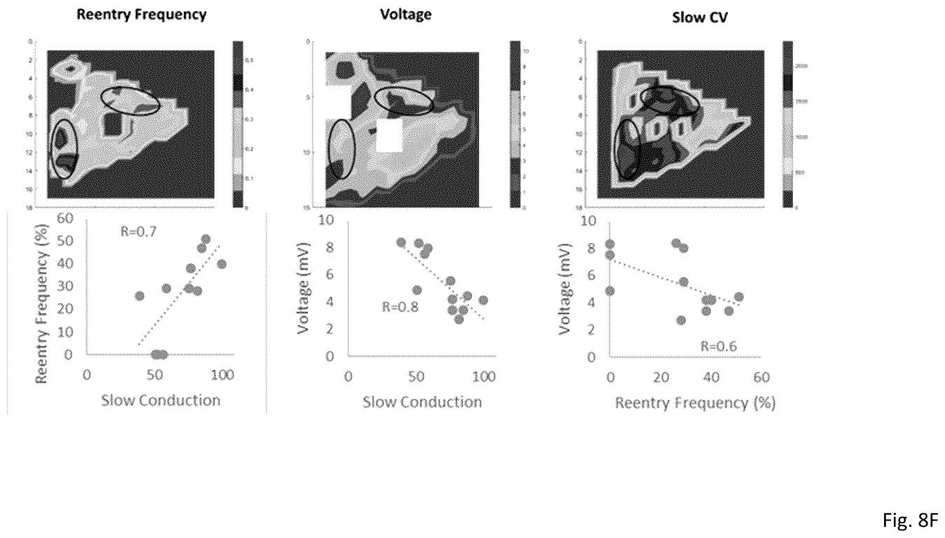

FIG. 8A depicts a 3D local activation time map in accordance with an illustrative embodiment. FIG. 8B depicts that ablation at a slowest reentry site terminated persistent AF in accordance with an illustrative embodiment. FIG. 8C depicts low voltage at the slowest reentry site in accordance with an illustrative embodiment. FIG. 8D depicts the lowest reentry site at the border between high and low entropy in accordance with an illustrative embodiment. FIG. 8E depicts how reentry and focal drivers had significantly lower voltage (critical AF zones) compared to not critical AF zones without reentry or focal drivers in accordance with an illustrative embodiment. FIG. 8F depicts reentry frequency at low voltage and slow conduction in dogs, correlation reentry with slow conduction and voltage, and correlation of slow conduction and voltage in accordance with an illustrative embodiment.

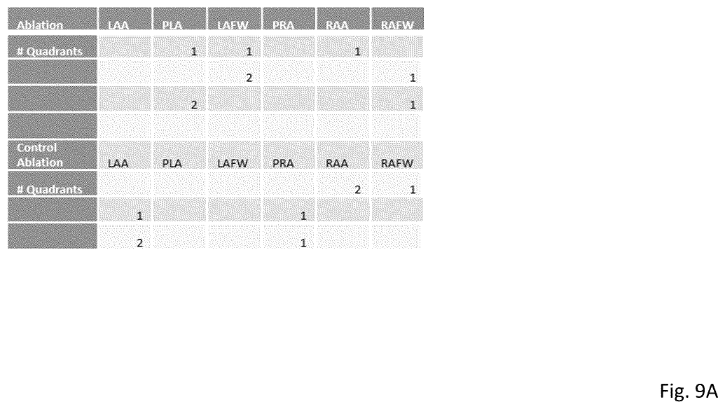

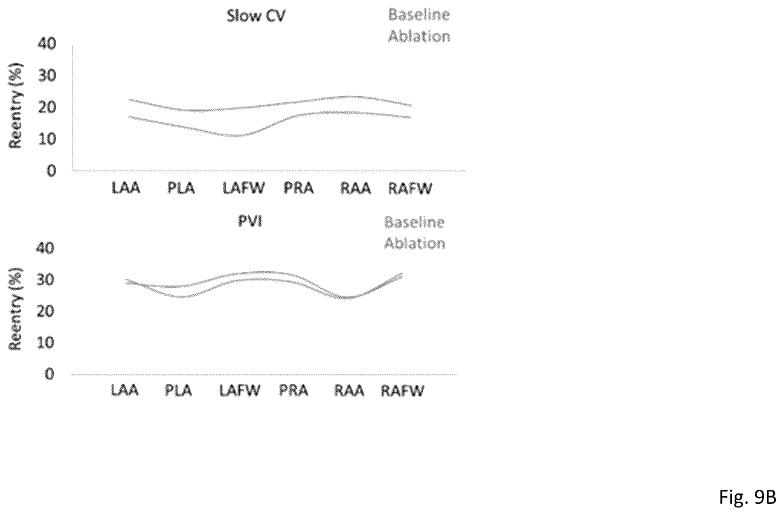

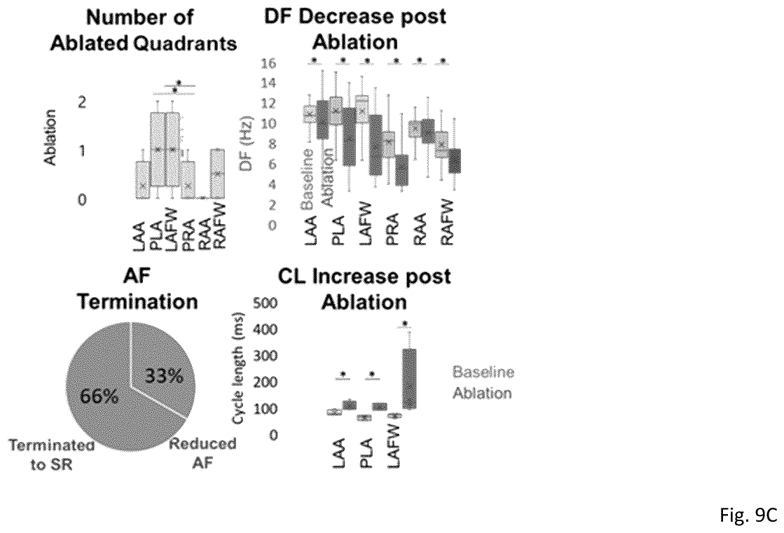

FIG. 9A is a chart that depicts the number of ablated mapping plaque quadrants with slowest reentry in the atrial regions and number of performed control quadrant ablations that lead to termination in 2 cases in accordance with an illustrative embodiment. FIG. 9B depicts that ablation at slow CV regions reduced observed reentry over time percentage in all regions in accordance with an illustrative embodiment. FIG. 9C depicts pulmonary vein isolation (PVI) reduced observed reentry over time percentage in all regions but not in the LAA and RAFW in accordance with an illustrative embodiment.

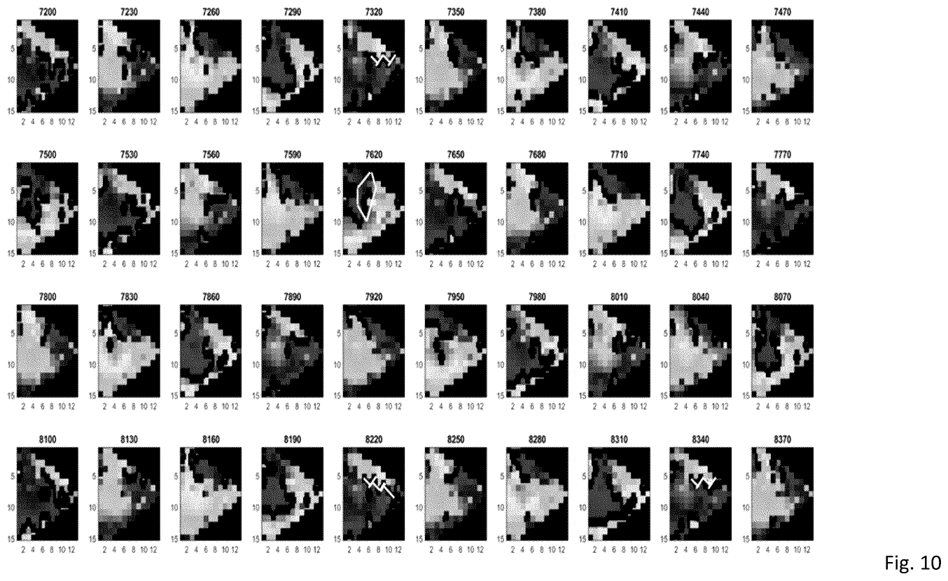

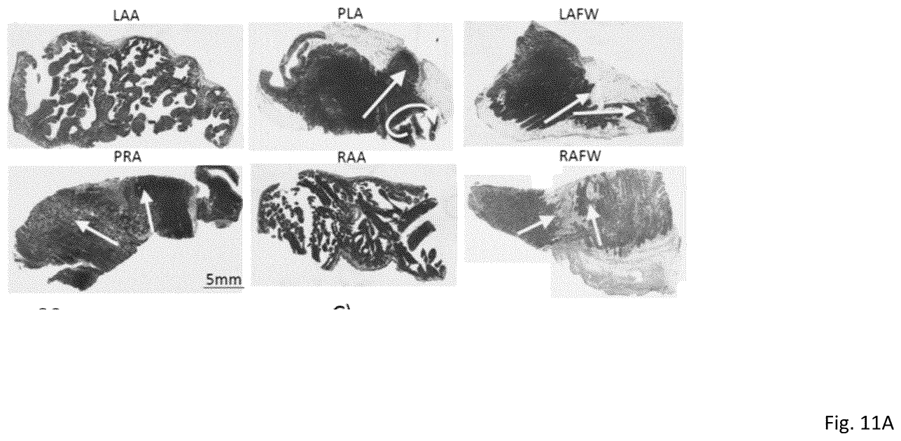

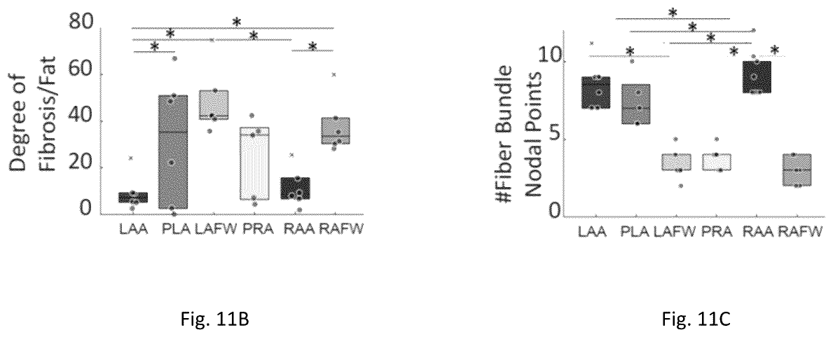

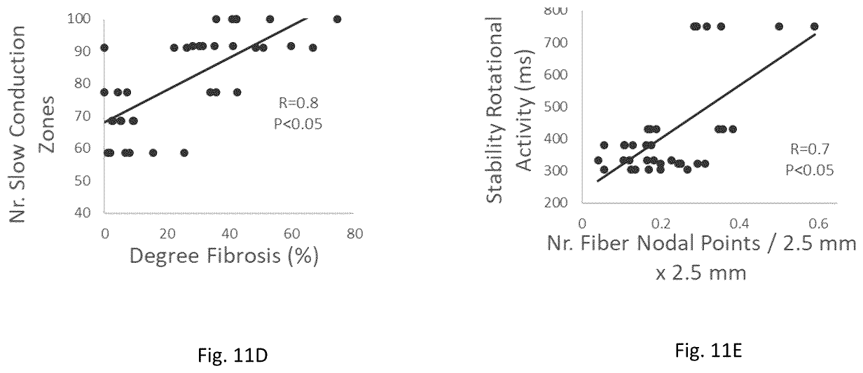

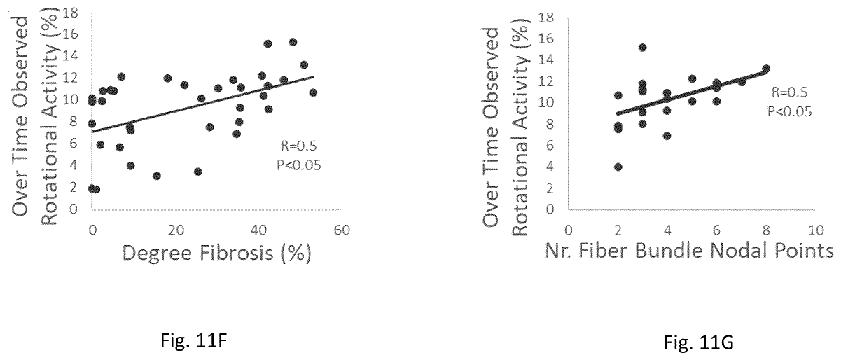

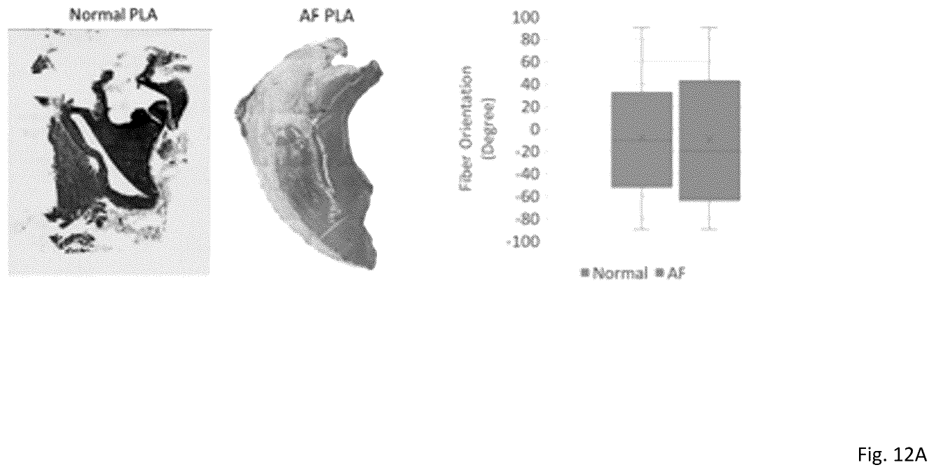

FIG. 10 depicts how block lines shift cycle by cycle but are similarly located and parallel over time in accordance with an illustrative embodiment. FIG. 10 also depicts how the block lines correlate with low voltage regions. FIG. 11 shows substrate characteristics and correlations with rotational activity and slow conduction zones. FIG. 11A depicts Masson's trichrome stained tissue sections in the six atrial regions in accordance with an illustrative embodiment. Fiber orientations are marked by the arrows in FIG. 11A. It is noted that dense focal fibrosis was located at myocyte bundle nodal points (intersections of fiber lines). FIG. 11B depicts the degree of focal fibrosis or fat in all atrial regions in both atria in accordance with an illustrative embodiment. The highest degree of fibrosis/fat was detected in the PLA and LAFW. FIG. 11C depicts the highest number of myocyte bundle nodal points that were detected in the RAA, LAA, and PLA in accordance with an illustrative embodiment. FIG. 11D depicts correlation of the degree of fibrosis/fat with detected slow conduction zones (activation delay zones>10 ms) (R=0.7, P<0.05) (N=35) in accordance with an illustrative embodiment. FIG. 11E depicts correlation of myocyte bundle nodal points with the stability of rotational activity (R=0.6, P<0.05) (N=35) in accordance with an illustrative embodiment. FIG. 11F depicts positive correlation between the observed rotational activity over time (%) and the degree of fibrosis in all atrial regions in accordance with an illustrative embodiment. FIG. 11G depicts positive correlation between the number of fiber bundle nodal points and the observed rotational activities over time in all regions but not the left and right atrial appendage in accordance with an illustrative embodiment. In FIG. 11, two-way ANOVA significance is indicated in graphs, with *P<0.05 for all comparisons.

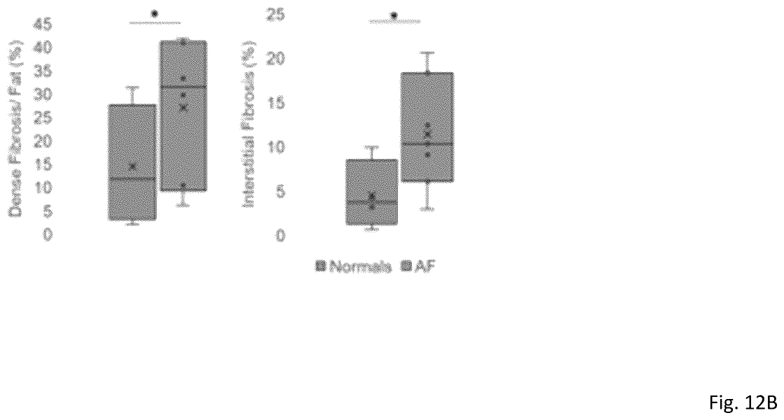

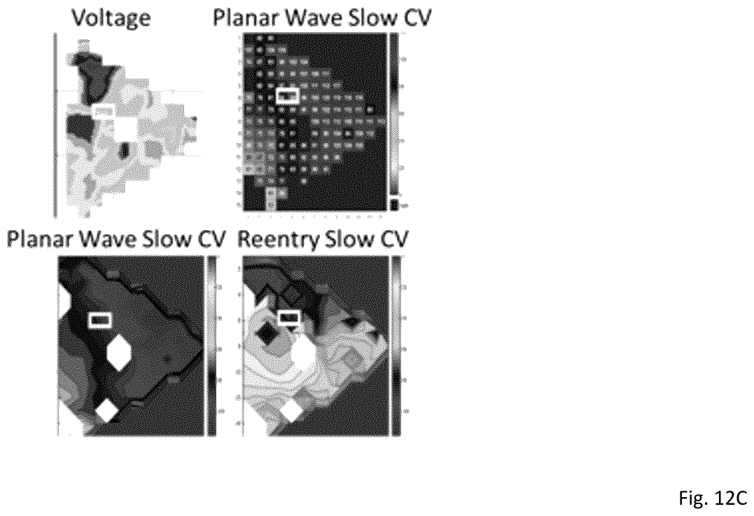

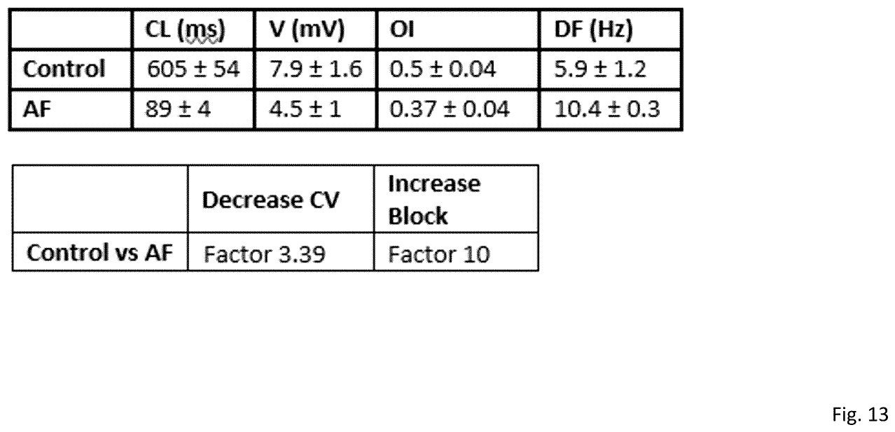

FIG. 12 shows how low voltage and slow conduction are correlated and can be identified in atrial fibrillation and normal sinus rhythm. Low voltage and slow conduction correlate with histological tissue fibrosis. Control animals conducted for fiber orientation, fatty and fibrotic tissue, regional conduction CV and slow conduction zones/blocks, AF CL. FIG. 12A depicts normal PLA versus AF PLA in accordance with an illustrative embodiment. FIG. 12B depicts dense fibrosis versus interstitial fibrosis in accordance with an illustrative embodiment. FIG. 12C depicts voltage, planar wave slow CV, and reentry slow CV in accordance with an illustrative embodiment. FIG. 13 includes tables comparing a control case versus an AF case in accordance with an illustrative embodiment.

In an illustrative embodiment, any of the operations described herein can be performed by a computing system that includes a memory, processor, user interface, network interface, display, etc. For example, any of the operations described herein can be implemented as computer-readable instructions stored on a computer-readable medium. Upon execution of the computer-readable instructions by the processor, the computing system performs the various operations described herein to implement the system. As an example, FIG. 14 depicts a computing device 1400 in direct or indirect communication with an electrode array 1435 in accordance with an illustrative embodiment. The computing device 1400 can also be connected to a treatment device such as a radiofrequency ablation (RFA) device (e.g., an RFA catheter), a cryoablation device, a laser device, an ablation chemical delivery device, etc. The computing device 1400 can be implemented as a dedicated computer that interacts with an electrode catheter system or other electrode array. In other embodiments, the computing device 1400 can be a smartphone, tablet, laptop computer, desktop computer, etc.

The computing device 1400 includes a processor (or microcontroller) 1405, an operating system 1410, a memory 1415, an input/output (I/O) system 1420, a network interface 1425, and an AF analysis application 1430. In alternative embodiments, the computing device 1400 may include fewer, additional, and/or different components. The components of the computing device 1400 communicate with one another via one or more buses or any other interconnect system.

The processor 1405 of the computing device 1400 can be in electrical communication with and used to control any of the systems described herein, such as the electrode array 1435, etc. The processor 1405 can be any type of computer processor known in the art, and can include a plurality of processors and/or a plurality of processing cores. The processor 1405 can include a controller, a microcontroller, an audio processor, a graphics processing unit, a hardware accelerator, a digital signal processor, etc. Additionally, the processor 1405 may be implemented as a complex instruction set computer processor, a reduced instruction set computer processor, an x86 instruction set computer processor, etc. The processor 1405 is used to run the operating system 1410, which can be a custom operating system specific to the requirements of the proposed system.

The operating system 1410 is stored in the memory 1415, which is also used to store programs, image data, algorithms, network and communications data, peripheral component data, and other operating instructions. The memory 1415 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 1420, or user interface, is the framework which enables users (and peripheral devices) to interact with the computing device 1400. The I/O system 1420 can include one or more keys or a keyboard, one or more buttons, one or more displays, a speaker, a microphone, etc. that allow the user to interact with and control the computing device 1400. The I/O system 1420 can also include a printer that prints out results of the analysis, such as a map of target locations for ablation, a description of the diagnosis, electrode data/results, etc. The I/O system 1420 further includes circuitry and a bus structure to interface with peripheral computing components such as power sources, sensors, etc.

The network interface 1425 includes transceiver circuitry that allows the computing device 1400 to transmit and receive data to/from other devices such as user device(s), remote computing systems, electrode systems, servers, websites, etc. The network interface 1425 enables communication through a network, which can be one or more communication networks. The network can include a cable network, a fiber network, a cellular network, a wi-fi network, a landline telephone network, a microwave network, a satellite network, etc. The network interface 1425 also includes circuitry to allow device-to-device communication such as near field communication (NFC), Bluetooth® communication, etc.

The AF analysis application 1430 can include hardware, software, and algorithms (e.g., in the form of computer-readable instructions) which, upon activation or execution by the processor 1405, performs any of the various operations described herein such as controlling an electrode array, receiving electrode data, performing signal processing on the received data, determining reentry rotation direction at a plurality of locations, identifying points of intersection of clockwise and anti-clockwise reentry rotations, identifying locations to perform ablation, controlling a machine (e.g., a laser) to perform the ablation at the identified target location(s), determining reentry percentage over time from a correlation between cycle length measurements and reentry percentage over time (FIG. 3D), determining the size including the length and the width of clockwise and anti-clockwise reentries (FIGS. 5A & 5B), determining the percentage of reentry degree completeness where 100% is associated with 360 degrees (FIG. 5D), determining and visualizing the length of the block that blocks atrial activation, defined as a >25 ms activation delay between electrodes with 2.5 mm electrode distances (FIG. 5E), determining and visualizing the gap length of block lines that block atrial activation, defined as a >25 ms activation delay between electrodes with 2.5 mm electrode distances (FIG. 5E), determining and visualizing atrial regions with high factors of clockwise vs anti-clockwise reentries and atrial regions with high factors of anti-clockwise vs clockwise reentries over time, i.e. factor>5 (FIG. 6J), determining reentry percentage over time from a correlation between activation delay between neighbored electrodes (e.g., 2.5 mm electrode distances) and reentry percentage over time (FIG. 6K), determining reentry percentage over time from a correlation between peak-to-peak amplitude and reentry percentage over time (FIG. 6K), determining peak-to-peak amplitude from a correlation between activation delay between neighbored electrodes (e.g., in 2.5 mm electrode distances) and peak-to-peak amplitude (FIG. 7), etc. The AF analysis application 1430 can utilize the processor 1405 and/or the memory 1415 as discussed above.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

1. A system to identify atrial fibrillation, the system comprising:
an electrode array that senses data from an atrium of a heart; and
a computing device operatively coupled to the electrode array, wherein the computing device includes a processor configured to:
identify, based on the sensed data from the electrode array, one or more first locations of the atrium at which a dominant direction of reentry is clockwise;
identify, based on the sensed data from the electrode array, one or more second locations of the atrium at which a dominant direction of reentry is anti-clockwise;
identify, based on analysis of the one or more first locations and the one or more second locations, an intersection point, wherein the intersection point is a location of the atrium at which there is both a dominant clockwise reentry and a dominant anti-clockwise reentry; and
determine a target location for treatment of the atrial fibrillation based on the intersection point.

2. The system of claim 1, wherein the processor further implements a treatment at the target location, wherein the treatment comprises an ablation.

3. The system of claim 1, wherein an interstitial spacing of the electrode array is 2.5 millimeters or less.

4. The system of claim 1, wherein the one or more first locations are identified based on a duration of time during which the dominant direction of rotation is clockwise.

5. The system of claim 1, wherein the intersection point is identified based on a duration of time during which there is both the dominant clockwise reentry and the dominant anti-clockwise reentry.

6. The system of claim 1, wherein the processor generates one or more heat maps based on the sensed data, and wherein the one or more first locations and the one or more second locations are identified based on the one or more heat maps.

7. The system of claim 1, wherein the processor is configured to determine reentry percentage over time based at least in part on a correlation between cycle length measurements and reentry percentage.

8. The system of claim 1, wherein the processor is configured to determine a size including a length and a width of one or more clockwise reentries and one or more anti-clockwise reentries.

9. The system of claim 1, wherein the processor is configured to determine a percentage of reentry degree completeness, wherein 100% is associated with 360 degrees.

10. The system of claim 1, wherein the processor is configured to determine and visualize a length of a block that blocks atrial activation.

11. The system of claim 1, wherein the processor is configured to determine and visualize a gap length of block lines that block atrial activation, wherein the block lines that block atrial fibrillation have greater than a 25 millisecond activation delay between electrodes with 2.5 millimeter electrode spacing.

12. The system of claim 1, wherein the processor is configured to determine and visualize atrial regions with high factors of clockwise versus anti-clockwise reentries and atrial regions with high factors of anti-clockwise versus clockwise reentries over time, wherein the high factors are greater than five factors.

13. The system of claim 1, wherein the processor is configured to determine reentry percentage over time based at least in part on a correlation between activation delay between neighboring electrodes and reentry percentage.

14. The system of claim 1, wherein the processor is configured to determine reentry percentage over time based at least in part on a correlation between peak-to-peak amplitude and reentry percentage.

15. A method of identifying atrial fibrillation, the method comprising:
receiving, at a memory of a computing device, data from an electrode array, wherein the data originates from an atrium of a heart;
identifying, by a processor of the computing device and based on the sensed data from the electrode array, one or more first locations of the atrium at which a dominant direction of reentry is clockwise;
identifying, by the processor and based on the sensed data from the electrode array, one or more second locations of the atrium at which a dominant direction of reentry is anti-clockwise;
identifying, by the processor and based on analysis of the one or more first locations and the one or more second locations, an intersection point, wherein the intersection point is a location of the atrium at which there is both a dominant clockwise reentry and a dominant anti-clockwise reentry; and
determining, by the processor, a target location for treatment of the atrial fibrillation based on the intersection point.

16. The method of claim 15, further comprising controlling, by the processor, a device to perform an ablation at the target location to treat the atrial fibrillation.

17. The method of claim 15, further comprising identifying the one or more first locations based on a duration of time during which the dominant direction of rotation is clockwise, wherein the duration of time is 10 seconds or less.

18. The method of claim 15, further comprising identifying the intersection point based at least in part on identification of a simultaneous occurrence of the dominant clockwise reentry and the dominant anti-clockwise reentry.

19. The method of claim 15, further comprising generating, by the processor, one or more heat maps based on the sensed data, and wherein the one or more first locations and the one or more second locations are identified based on the one or more heat maps.

20. The method of claim 15, further comprising determining, by the processor, reentry percentage over time based at least in part on activation delay between neighboring electrodes in the electrode array.
